(19) United States
(12) Patent Application Publication
(10) Pub. No.: US 2003/0095997 A1
(43) Pub. Date: May 22, 2003

(54) Natural polymer-based material for use in human and veterinary medicine and method of manufacturing

(76) Inventors: Zbigniew Ruszczak, Munchen (DE); Robert Mehrl, Langquald (DE); Johann Jeckle, Riedenburg (DE)

Correspondence Address: Synnestvedt & Lechner LLP, 2600 Aramark Tower, 1101 Market Street, Philadelphia, PA, 19107-2950, US

(21) Appl. No.: 10/231,628

(22) Filed: Aug. 30, 2002

Related U.S. Application Data
(63) Continuation-in-part of application No. PCT/EP00/02056, filed on Mar. 9, 2000.

(51) Int. Cl. A61K 9/70; C12N 5/02

(52) U.S. Cl. 424/443; 442/327; 435/402

(57) ABSTRACT

The invention is concerned with relatively dense and fluid expandable natural polymer-based membrane, preferably collagen-based material, with improved mechanical, physical, functional, and handling properties for use in human and veterinary medicinal applications. The membranous material is preferably collagen, and optionally contains additional biologically active substances such as hemostatic agents, growth factors, cytokines, hormones, drugs and the like, and/or biologically important and tissue-compatible inorganic or/and organic substances or/and their derivatives which can improve the mechanical, functional and handling properties of the material. The membrane is obtainable by simultaneous heat and pressure treatment of a basic natural polymer material for short periods of time.

This application is a continuation-in-part application of PCT/EP00/02056 designating the US, filed Mar. 9, 2000.

### FIELD OF THE INVENTION

The present invention is concerned with natural polymer sponges and membranes, for use in medical and surgical applications, tissue regeneration implants, hemostatic agents, drug delivery systems, and wound healing materials. The present invention relates more particularly to novel natural polymer-based materials with improved mechanical, physical, functional, and handling properties for the aforesaid and to methods for the manufacture thereof.

Dressings and implants for use in wound healing should have the ability to adhere and conform to the wound site. Such materials ideally should facilitate regrowth of tissue, by virtue of the accumulation of fibroblasts, endothelial cells, and wound healing regulatory cells into the wound site. Such accumulation of cells promotes connective tissue deposition and angiogenesis and speeds healing. The chemical composition and physical characteristics of the implant or dressing are critical to whether these objectives are realized.

Collagen is a major substituent of certain membranes surrounding important organs and separating different tissues and cells, and acts as a superstructure on which cells proliferate, in humans and other animals. Examples of large membranes include the pericardium, peritoneum, intestinal and placental membranes while on the microscopic level, examples include the basal membranes. Consequently, collagen, the major protein of connective tissue, is used in wound dressings and surgical implants

Various different xenogenous, allogenic or autologous collagen-based materials are used in human and veterinary medicine. Purified collagen, even of xenogenous origin, is almost fully biocompatible with human (and also animal of different species) collagenous tissue and may be incorporated into and/or subsequently remodeled to a host connective tissue without foreign body reaction and immunologic rejection. Procedures for rendering xenogeneic collagen substantially non-immunogenic are available. A variety of collagen forms are available including soluble collagen, collagen fibers, collagen processed into sponges, membranes and bone implants. For example, collagen fibers and sponges are used for haemostasis, tissue augmentation and/or as carriers for biologically active substances, collagen membranes are used for wound covering or implantation, as substitutes for missing tissue such as skin, injections of soluble collagen are used in plastic surgery, and multilayer collagen implants based on processed animal large membrane are used for the above applications as well as guided tissue regeneration.

Collagen-based hemostatic agents must have both biological and mechanical features promoting haemostasis such as intact native collagen fibers and optimal porosity. For use as a tissue substitute or equivalent, the collagen-based material must have optimal matrix properties promoting cell growth, formation of granulation tissue, angiogenesis, and vascularization. Collagen-based carriers of biologically active substances must have features allowing an optimal release and pharmacokinetics of the incorporated active substance.

Collagen-based membranes used in surgeries to guide tissue regeneration must have appropriate biological and physical characteristics beyond the few mentioned above. Following surgeries, where wound healing is desirable, undesirable tissue in-growth complicates appropriate tissue regeneration. For example, in dental surgery where a substantial portion of a tooth root is removed, the desired result is the regeneration of healthy bone tissue to replace the bone tissue removed. However, absent appropriate intervention, the cavity left by removal of the bone fills with connective tissue effectively preventing bone regeneration. To prevent this process from delaying healing, a membrane is surgically inserted around the periphery of the wound cavity. This membrane must deter adventitious cell infiltration of the wound cavity and permit the growth of desirable cells.

In all cases, the handling properties of the collagen-based material, including its mechanical strength and stability, its flexibility and, if necessary, its ability to be sutured or sealed are of practical importance.

Reported Developments

Commercially collagen-based materials are available in the form of sponges, transparent membranes, multilayer animal membrane based products, and injectable solutions of varying viscosities. Collagen-based sponges and membranes are used for tissue substitution, haemostasis, skin substitution and as a carrier for biologically active substances. The Collatamp®-G product, manufactured by SYNTACOLL AG, Herisau, Switzerland, is a gentamycin-containing sponge that is sold and distributed worldwide by Schering-Plough and affiliates, and is the only commercially available collagen-based drug delivery system for antibiotics.

Various procedures have been described to improve the mechanical properties of collagen materials. The use of mechanical pressure at ambient temperature for industrial manufacture of freeze-dried collagen sponges, such as sponges containing gentamycin antibiotic is known (e.g., EP 0069260, issued Sep. 25, 1985, owned by Syntacoll AG, Herisau, Switzerland). Heating reconstituted collagen membrane-like products is known as a curing mechanism, and may initiate cross-linking of the collagen, but can be time consuming and injurious to the native fibril structure of the collagen. “Dehydro-thermal treatment” uses negative air pressure to drive such cross-linking processes. Additional procedures include additional cross-linking procedures, the most popular of which are chemical cross-linking, for example with aldehydes. The aldehyde-based cross-linking is capable of negatively influencing the biocompatibility of collagen and lead to residual aldehyde, or aldehyde derivatives, in the cross-linked product.

U.S. Pat. No. 3,157,524 discloses a sponge comprised of acid treated swollen collagen. Oluwasanmi et al. (J. Trauma 16:348-353 (1976)) discloses a 1.7-millimeter thick collagen sponge that is cross-linked by glutaraldehyde. Collins et al. (Surg. Forum 27:551-553 (1976)) discloses an acid-swollen collagen sponge that is cross-linked by glutaraldehyde. U.S. Pat. No. 4,320,201 discloses a swollen sponge of high collagen purity produced by enzymatically degrading animal hides, digesting the mass in alkali or acid, mechanically comminuting the mass to produce specified lengths of collagen fibers, and cross linking the fibers. U.S. Pat. No. 4,837,285 discloses porous beads that have a collagen skeleton of 1 to 30 percent of the bead volume. These beads are useful as substrates for cell growth. In addition, collagen has been used as a component in salves (PCT Patent Application WO 86/03122). U.S. Pat. No. 4,937,323 discloses the use of collagen for wound healing in conjunction with electrical currents. Abbenhaus et al., Surg. Forum 16:477-478 (1965) discloses collagen films of two to three millimeter thickness that were produced by heating and dehydrating collagen extracted from cow hides. U.S. Pat. No. 4,412,947 discloses an essentially pure collagen sheet made by freeze-drying a suspension of collagen in an organic acid. British Patent 1,347,582 discloses a collagen wound dressing consisting of a freeze-dried polydisperse collagen mixture. U.S. Pat. No. 4,950,699 discloses a wound dressing consisting of less than 10% collagen mixed with an acrylic adhesive.

European Patent Application 187014, U.S. Pat. Nos. 4,600,533; 4,689,399; and PCT Patent Application WO 90/00060 disclose non-chemically cross linked collagen implants produced by compression, which are useful for sustained drug delivery. U.S. Pat. No. 4,453,939 discloses a wound-healing composition containing collagen coated with fibrinogen, factor XIII fibrinogen, and/or thrombin. U.S. Pat. No. 4,808,402 discloses a composition for treating wounds comprising collagen, bioerodible polymer, and tumor necrosis factor. U.S. Pat. No. 4,703,108 discloses that fibronectin, laminin, type IV collagen and complexes of hyaluronate and proteoglycans may be included in a collagen-based matrix, having a swelling ratio of between 2.5 to 5 for collagen-based matrices that comes into contact with open wounds, or a swelling ratio of between 2.5 to 10 for collagen-based matrices for subcutaneous implantation. The thickness of the collagen-based matrix is varied from 1 to several hundred mm, and preferably between 2 to 3 mm for full thickness wound dressings.

Most currently available collagen-based materials prepared from reconstituted collagen are, however, not stable enough to be sutured, rolled, or stitched, especially in areas of mechanical tension or in difficult anatomical sites. Moreover, collagen sponges or membranes are, in many cases, not strong enough to sufficiently cover defects of such tissue including dura matter, superficial and deep skin wounds, bones, and nerves.

U.S. Pat. No. 4,522,753 describes a method for preserving porosity and improving stability of collagen sponges by both aldehyde and dehydro-thermal treatment. The negative pressure (vacuum) used in this process may vary from about 1 mtorr up to a slight vacuum below atmospheric pressure. U.S. Pat. No. 4,578,067 describes a hemostatic-adhesive collagen dressing in the form of a dry-laid, non-woven, self-supporting web of collagen fiber. The manufacturing of such material is based on a Rando-feeder and Rando-webber techniques. The collagen fibers from the Rando-feeder are introduced into the air stream of the Rando-webber and form a fiber mass of uniform density. Such mass is then processed by pressing or embossing or by calendaring at a temperature ranging from room temperature to 95° C. The inherent limitation of such techniques is that the pressures to which the fiber mass is subjected are limited to preparing relatively thick layers of material of relatively low density.

U.S. Pat. No. 5,206,028 describes a collagen membrane that does not swell appreciably upon being wetted and maintains its density. The manufacturing of such translucent, collagen Type-1 based material is based on compression of collagen sponges in a roller press with a calibrate aperture followed by aldehyde cross-linking. For additional mechanical stabilization, the cross-linked membrane may be re-wetted, re-lyophilized, and pressed again under standard condition.

U.S. Pat. No. 5,567,806 discloses suturable, biocompatible, control-resorbing membranes for use in guided tissue regeneration, comprising a cross-linked collagen material either obtained by cross linking a starting collagen material in the coagulated state or obtained by cross linking a sponge of chondroitin 4-sulfate added to 0.75% collagen gel material that has been compressed under a pressure of 150 bars, and on which a collagen material gel has been poured before performing the cross linking. The '806 patent neither suggests or discloses simultaneous heating and pressure treatment of a collagen material.

U.S. Pat. No. 4,948,540 describes a mechanically stable, collagen wound dressing sheet material fabricated by lyophilizing a collagen composition and compressing the porous pad at a pressure between about 15,000 and 30,000 p.s.i to a thickness of between 0.1 to 0.5 centimeters (1 to 5 mm) at a pressure to yield a collagen dressing sheet material having an absorbability of 15-20 times its weight. The '540 patent also discloses that the material may be cross-linked by dehydro-thermal treatment to improve mechanical stability.

U.S. Pat. No. 4,655,980 discloses the manufacturing of collagen membrane articles based on a soluble collagen gel suspension. The membrane may be obtained by applying pressure to the gel, or by disrupting the gel and separating the resulting precipitate for casting. Depending on the dimension and shape of the casting mold, either a membrane or solid can be obtained. The manufacturing of such membrane is based on a commercially available soluble, injectable, atelocollagen product of Collagen Aesthetics, Palo Alto, Calif., USA.

U.S. Pat. No. 5,219,576 and WO99/19005 describe a collagen implant material useful as a wound healing matrix and delivery system for bioactive agents. The '576 patent discloses the manufacturing of multilayer collagen materials by serially casting and freezing the individual layers and then lyophilizing the entire composite at once. Additional cross-linking by both aldehyde and dehydro-thermal processing of the final product is also disclosed. The '576 patent discloses compressing the single layer implants from a thickness of 5 mm to 1 mm to increase its bulk density. The '576 patent discloses that compressed implants typically have bulk densities in the range of 0.05 to 0.3 g/cc, whereas non-compressed implants normally have bulk densities of 0.01 to 0.05 g/cc. The '576 patent does not suggest simultaneous heat curing and compression.

There is a need, however, both in human and veterinary medicine, for a practical, efficient and industrially manufactured, ready-to-use, user-friendly collagen-based material, or materials based on other natural polymers showing similar properties, which are fully biocompatible, mechanically stable, flexible, easy-to-handle, and which can be sutured, sealed, rolled, screwed, cut and/or meshed. Moreover, it would be of advantage, if such material incorporated biologically active substances to promote healing, stimulate organ reconstitution and treat or prevent infections.

The present invention, therefore, provides a novel natural polymer-based product with improved mechanical, physical, and bio-physiological properties that can be easily manufactured by available industrial methods. The present invention further makes available new options for the use of reconstituted collagen-based materials in medical and surgical applications for which the use of such prior art biological material was previously neither possible nor practical. Furthermore, the present invention provides a platform for the development of further implant and wound-dressing constructions, which could not be efficiently, manufactured with prior art materials.

### SUMMARY OF THE INVENTION

The present invention relates to a biocompatible membrane comprising a reconstituted matrix of natural polymer fibers and fibrils in their native or re-natured form, wherein said membrane is less than one mm in thickness, has a collagen density of about 250 mg/cm3 to about 500 mg/cm3, and is capable of expanding in contact with fluids to form a matrix capable of promoting cell growth. Further aspects of the present invention relate to embodiments wherein said expanded matrix is porous and capable of promoting the formation of granulation tissue, angiogenesis, vascularization, and epithelization. The preferred membrane comprises collagen that exhibits the hemostatic and non-antigenic properties of native collagen.

A further aspect of the present invention is a process for the preparation of a biocompatible membrane comprising a reconstituted matrix of natural polymer fibers and fibrils and having improved mechanical and fluid absorption properties, said process comprising applying pressure and heat to a collagen sponge to reduce the thickness of said sponge to about 1 to about 30 percent of the thickness of said sponge, for a time sufficient to improve said mechanical properties and to preserve the native and/or re-natured form of said fibers and fibrils. The present process enables the preparation of such membranes having improved mechanical properties such as dry and wet tensile strength, suturing and wetting ability, and flexibility such that said membrane is capable of being rolled, screwed in both dry and wet condition, cut and meshed without breaking or deforming.

Further preferred aspects of the present invention are further described in more detail below.

### DETAILED DESCRIPTION

The terms defined in this section are used throughout this specification.

The term “antibiotic” as used herein means a substance produced synthetically or isolated from natural sources that selectively inhibits the growth of a microorganism.

The term “biocompatible” as used herein means the ability of a material to pass the biocompatibility tests set forth in International Standards Organization (ISO) Standard No. 10993 and/or the U.S. Pharmacopoeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” These tests assay as to a material's toxicity, infectivity, pyrogenicity, irritation potential, reactivity, hemolytic activity, carcinogenicity, and/or immunogenicity. A biocompatible membrane, or polymer comprising a membrane, when introduced into a majority of patients will not cause an adverse reaction or response. In addition, it is contemplated that biocompatibility can be effected by other contaminants such as prions, surfactants, oligonucleotides, and other biocompatibility effecting agents or contaminants.

The term “contaminant” as used herein means an unwanted substance on, attached to, or within a material, such a layer of the present invention. This includes, but is not limited to bioburden, endotoxins, processing agents such as antimicrobial agents, blood, blood components, viruses, DNA, RNA, spores, fragments of unwanted tissue layers, cellular debris, and mucosa.

The term “cells” as used herein means a single unit biological organism that may be eukaryotic or prokaryotic. The eukaryotic cell family includes yeasts and animal cells, including mammalian and human cells. Cells that may be useful in conjunction with the present invention include cells that may be obtained from a patient, or a matched donor, and used to seed a wound site. Such seeding would be used in an effort to repopulate the wound area with specialized cells, such as dermal, epidermal, epithelial, muscle or other cells, or alternatively to provide cells those stimulates or are involved in providing immunological protection to fight off infectious organisms. Such cells may be isolated and extracted from the patient, and/or genetically reengineered to produce a host of cytokines, antibodies, or other growth factors to aid in the wound healing process.

The term “cytokine” as used herein means a small protein released by cells that has a specific effect on the interactions between cells, on communications between cells or on the behavior of cells. The cytokines includes the interleukins, lymphokines, and cell signal molecules, such as tumor necrosis factor and the interferons, which trigger inflammation and respond to infections. Many cytokines are produced by recombinant technology and are presently available for use in research as well as by prescription in human and animal subjects.

The term “growth factor” as used herein means a substance (as a vitamin B₁₂ or an interleukin) that promotes growth and especially cellular growth. Examples of growth factors include, but are not limited to, epidermal growth factor, which is a polypeptide hormone that stimulates cell proliferation, nerve growth factor, which is a protein that promotes development of the sensory and sympathetic nervous systems and is required for maintenance of sympathetic neurons, vascular endothelial growth factors, which are a family of proteins that stimulate angiogenesis by promoting the growth of vascular endothelial cells, and the like. The term “oncostatically effective amount” is that amount of growth factor that is capable of inhibiting tumor cell growth in a subject having tumor cells sensitive to the selected agent. For example, many non-myeloid carcinomas are sensitive to treatment with TGF-beta, particularly TGF-beta2. The term “hematopoietically modulatory amount” is that amount of growth factor that enhances or inhibits the production and/or maturation of blood cells. For example, erythropoietin is known to exhibit an enhancing activity at known dosages, while TGF-beta exhibits an inhibitory effect. The term “osteoinductive amount” of a biological growth factor is that amount which causes or contributes to a measurable increase in bone growth, or rate of bone growth.

The term “medicament” as used herein means a substance used in medical therapy, such as the therapeutically effective active ingredient in a pharmaceutical. The term “immunomodulatory amount” of a medicament or agent is an amount of a particular agent sufficient to show a demonstrable effect on the subject's immune system.

Typically, immunomodulation is employed to suppress the immune system, e.g., following an organ transplant, or for treatment of autoimmune disease (e.g., lupus, autoimmune arthritis, autoimmune diabetes, etc.). For example, when transplanting an organ one could line the site with the matrix of the invention impregnated with an immunomodulatory amount of an immunosuppressive biological growth factor to help suppress rejection of the transplanted organ by the immune system. Alternatively, immunomodulation may enhance the immune system, for example, in the treatment of cancer or serious infection (e.g., by administration of TNF, IFNs, etc.).

The term “membrane” as used herein means a thin soft pliable sheet or layer.

The term “natural polymer” as used herein means a polymer that is found in nature and that may be derived from natural sources or produced synthetically. More particularly, the natural polymer means a polymer comprising repeating subunits of small organic molecules found in biological systems including microorganisms, plants, and animals. Exemplary subunit molecules include the groups of molecules known as the nucleotides, amino acids, and saccharide molecules. Polymers containing these small molecules comprise the polynucleic acids, such as the polyribonucleic acids and the polydeoxyribonucleic acids, the polypeptides, such as the proteins including the structural proteins collagen, hyaluronic acid and keratin, and small polypeptides comprising certain hormones and other signaling molecules, and polysaccharides, such as the cellulose and alginic acid family of molecules, respectively.

Preferred natural polymers exhibit properties similar to collagen and are useful for the same applications. Examples of such substances are collagen and hyaluronic acid. Collagen is the more preferred natural polymer.

The collagen used for manufacture of the collagen-based materials of the present invention may be either of animal origin (xenogenous to humans) or human origin (autologous or allogenic) or may be obtained from genetically manipulated organisms (recombinant techniques and/or transgenic organisms), or by any other similar or/and equivalent methods. The collagen used for manufacturing of the improved collagen-based material may be of Type-I, Type-II, Type-III, Type-IV, Type-VII, or Type-IX alone or may be a mixture of two or more of such collagens. The more preferred collagen used for manufacture of the present collagen-based multilayer product is Type-1 collagen. This material can be easily obtained from animal tissue, such as skin, tendons, and membranes, by industrial methods know to the person of skill in the art, in accordance with GMP standards of manufacturing.

The present invention may use enzymatically treated collagen or collagen that has not been enzymatically treated. Preferred collagen is enzymatically treated with proteolytic enzymes, to separate the non-helical parts of the collagen molecule (telopeptides) from the triple-helical collagen chain (atelocollagen). Although in the examples collagen is described as the natural polymer material, within the framework of the invention it is to be understood that the same description of manufacture and physical properties of the final product also applies to other natural polymers that satisfy the above definition.

The term “reconstituted” as used herein describes a material that has its origin in a solid source or form such as a solid matrix, that has been disrupted by chemical, physical or biological processes, that may have been dispersed or dissolved in a liquid medium, and that has been reformed, or restructured, into a further solid form having a structure that is modified physically and/or chemically relative to the original solid form of the material.

The reconstituted natural polymer matrices and/or the synthetic polymer material layer may optionally contain biologically active substances such as hemostatic agents or tissue sealants, such as polysaccharides and glycosaminogtycans, proteins, such as fibrinogen, fibrin, cytokines and growth factors and hormones, cells or cell extracts medicaments, such as antibiotics, anti-inflammatory agents, or biologically important and tissue-compatible inorganic or/and organic substances or/and their derivatives which can improve the mechanical, functional, biological and handling properties of the material. Exemplary antibiotics include but are not limited to gentamycin, tetracycline, doxycycline, teicoplanin, quinoline antibiotics including the fluroquinolones, vancomycin, synercid®, penicillin derivatives and the cephlosporins. One or more protein agents may be incorporated to promote granulation tissue deposition, angiogenesis, re-epithelialization, and fibroplasia. Additionally, these and other factors are known to be effective immunomodulators (either locally or systemically), hematopoietic modulators, osteoinductive agents, and oncostatic agents (e.g., TGF-beta has been shown to exhibit all of these activities). The bioactive additives or protein factors used herein may be native or synthetic (recombinant), and may be of human or other mammalian type. Human FGF (including both acidic or basic forms), PDGF, and TGF-beta are preferred. Methods for isolating FGF from native sources (e.g., pituitary, brain tissue) are described in Bohlen et al, Proc Nat Acad Sci USA, (1984) 81:5364, and methods for isolating PDGF from platelets are described by Rainer et al, J Biol Chem (1982) 257:5154. Kelly et al, EMBO J (1985) 4:3399 discloses procedures for making recombinant forms of PDGF. Methods for isolating TGF-beta from human sources (platelets and placenta) are described by Frolik et al in EPO 128,849 (Dec. 19, 1984). Methods for isolating TGF-beta and TGF-beta2 from bovine sources are described by Seyedin et al, EPO 169,016 (Jan. 22, 1986), and U.S. Ser. No. 129,864, incorporated herein by reference. Other exemplary agents include, without limitation, transforming growth factor-alpha, beta-thromboglobulin, insulin-like growth factors (IGFs), tumor necrosis factors (TNFs), interleukins (e.g., IL-1, IL-2, etc.), colony stimulating factors (e.g., G-CSF, GM-CSF, erythropoietin, etc.), nerve growth factor (NGF), and interferons (e.g., IFN-alpha, IFN-beta, IFN-gamma, etc.). Synthetic analogs of the factors, including small molecular weight domains, may be used provided they exhibit substantially the same type of activity as the native molecule. Such analogs are intended to be within the scope of the term “wound healing agent,” as well as within the specific terms used to denote particular factors, e.g., “FGF,” “PDGF,” and “TGF-beta.” Such analogs may be made by conventional genetic engineering techniques, such as via expression of synthetic genes or by expression of genes altered by site-specific mutagenesis. Factors, such as with PDGF, may be incorporated into the native polymer layer in its native form (i.e., in platelets), or as crude or partially purified releasates or extracts. Alternatively, the factors may be incorporated in a substantially pure form free of significant amounts of other contaminating materials.

Such additional agents are included in the reconstituted natural polymer layer in therapeutically effective local concentration amounts. The amount of the agent included in the material of the present invention will depend upon the particular agent involved, its specific activity, the type of condition to be treated, the age and condition of the subject, the severity of the condition and intended therapeutic effect. For example, it may be necessary to administer a higher dosage of a factor when using the present material to treat a wound resulting from surgical excision of a tumor, than when simply promoting the healing of a wound (e.g., due to trauma or surgical procedure). In most instances, the protein factor(s) will be present in amounts in the range of about 3 ng/mg to 30 ug/mg based on weight of collagen. Antibiotic agents, such as gentamycin, are present in amounts that range from about 100 microgram/cm3 to about 1×10⁴ microgram/cm3.

The present invention provides materials comprising reconstituted natural polymer matrices, preferably collagen-based matrices, that are biocompatible, resorbable, and that exhibit improved and variable, but defined, mechanical stability, dry and wet tension, fluid absorption, and flexibility.

The preferred membranes according to the present invention comprise collagen that exhibits the hemostatic and non-antigenic properties of native collagen. Such collagen is biocompatible, biodegradable, and resorbable. The membranes of the present invention are preferably prepared from reconstituted collagen matrix, and more preferably from reconstituted collagen matrix sponges. The term “sponge” as used herein means an elastic porous mass of interlacing fibers that is able when wetted to absorb water. The term “pore” as used herein means a small interstice admitting the absorption or passage of liquid or a cell. “Porous” means a material containing pores.

Another aspect of the present invention is a membrane having a wet tensile strength such that the material, alone or when used in a multilayer composite, may be handled wet during surgical procedures and sutured without tearing under normal conditions of use. A more preferred membrane exhibits a wet tensile strength of about 2 N to about 3 N.

Preferred membranes of the present invention have a thickness of less than about 1 mm, and a density of about 250 mg/cc to about 500 mg/cc. More preferred aspects include membranes having thickness of from about 0.01 to about 0.9 mm. Other embodiments of the preferred reconstituted membrane preferably have a thickness from about 0.05 mm to about 0.1 mm. A most preferred membrane has a density of about 250 mg/cm3 to about 300 mg/cm3.

A particularly preferred membrane according to the present invention includes a matrix that is capable of absorbing from about 3 to about 30 times its weight in fluids. A special embodiment of the membrane is capable of absorbing from about 20 to about 30 times its weight in fluids. Such expanded reconstituted membranes provide for pore sizes that permit the passage of cells that contribute to tissue regeneration and wound healing. A further special embodiment of the present invention comprises a membrane is capable of absorbing from about 3 to about 10 times its weight in fluids. Such expanded layers provide for pore sizes that permit the passage of cells at a slower rate, and consequently the resorbtion and bio-degradation of such denser, less expandable membranes is slower. The smaller the fluid expansion, the smaller the pore size, and the more impenetrable the particular layer will be to the passage of cells and biological fluids.

The present invention further provides a process for the preparation of a biocompatible membrane having sufficient flexibility useful for tissue and organ reconstruction. Preferably, the present process produces a biocompatible membrane comprising a reconstituted matrix of collagen fibers and fibrils and having improved mechanical and fluid absorption properties, said process comprising applying pressure and heat to a collagen sponge to reduce the thickness of said sponge to about 1 to about 30 percent of the thickness of said sponge, for a time sufficient to improve said mechanical properties and to preserve the native and/or re-natured form of said collagen fibers and fibrils. The present invention enables the manufacture of the present improved product under conditions that protect the fibril native (and/or re-natured) structure of the reconstituted collagen from degradation, denaturation, and melting and that preserves the natural biologic properties of collagen, including the hemostatic properties and non-antigenic properties.

The temperature used in the present process is in a range of from about 50° C. to about 200° C., while the pressure used is in a range from about 0.1 to about 1000 kg/cm². The time during which the thermal compression is administered to the material is between 0.1 second to about one hour. A preferred process according to the present invention employs a pressure of about 5 to about 25, and more preferably from about 5 to about 10 kg/cm2, an elevated temperature from about 50 to about 140° C., and a thermal compression time of about one to about 60 seconds. A more preferred treatment time is about 5 to about 10 seconds. A more preferred temperature is about 80 to about 140 degrees C. A further preferred thermal compression time is from about ten to about 30 seconds, and a most preferred time is about 10 to about 20 seconds.

The present process according to the invention preferably uses an uncompressed sheet that comprises a biocompatible collagen sponge having a thickness of about 1 to about 10 mm, and a density of about 2 to about 60 mg/cc. A preferred membrane is prepared by applying pressure and heat to a collagen sponge having a thickness of about 5 mm and a density of about 5 mg/cm3 to about 50 mg/cm3. A more preferred sponge used in the present process has a density between about 6 to about 30 mg/cm3. Preferably, according to the present invention, the water or solvent content of the sponge ranges from 2% to 40% of weight, and more preferably from about 10 to about 18% by weight.

A further aspect of the present invention is a membrane prepared by the present process invention wherein the thickness of said uncompressed sheet is reduced to about 1 to about 30 percent of its original thickness. A preferred embodiment of the process is the preparation of a membrane where the thickness is reduced to about 1 to about 15 percent of its original thickness. A special embodiment of the invention is where the thickness is reduced from about one to about 3 percent of its original thickness, and the resulting membrane is capable of absorbing about 3 to about 7 times its weight in fluids in about one hour. A more preferred embodiment is where the thickness of said layer is reduced to about 5 to about 30 percent of its original thickness, and is capable of absorbing about 4 to about 30 times its weight in fluids in about one hour. A most preferred embodiment according to the present invention is wherein the thickness of said layer is reduced to about 6 to about 25 percent of its original thickness, and is capable of absorbing about 25 to about 30 times its weight in fluids in about one hour.

The present process increases the density of a collagen sponge by about 8 to about 50 times of its original density.

The uncompressed collagen sponge layer may be manufactured using various state-of-the-art techniques. The material is preferably cast from a collagen dispersion/suspension (i.e. in water or other non-organic solvent) containing from about 0.5 to about 5 weight % of dry collagen The sponge is prepared preferably by freeze-drying the cast dispersion.

The treatment can be conducted in a conventional thermal pressing machine in which the parts exerting the pressure can be adjusted to a predefined and constant temperature. The manufacturing steps used for the preparation of the novel material can be easily incorporated into routine manufacturing processes and allows the savings of time and costs compared to other currently used methods used for the production of collagen products.

As a result of such a heat and pressure treatment, a biocompatible collagen-containing membrane-like structure of desired thickness, mechanical strength, permeability, degradation and resorption time, can be manufactured. Moreover, the manufactured product exhibits much better handling properties, including rolling, screwing, and suturing, than other known collagen-based products such as freeze-dried sponges or air-dried membranes. Specifically, the improved properties of the material of the invention allow the use of sutures and other methods of mechanical fixation in situ, which use was not possible in the case of traditional reconstituted collagen-based materials. Moreover, through application of the present processing invention, the materials permeability for air (or other gases) and water (or other fluids, including blood, or tissue fluids) as well as mechanical strength can easily be controlled due to variations in the manufacturing process. The variations of the processing parameters to achieve such varying properties are known to the skilled artisan.

Additionally the compressed reconstituted and biocompatible collagen-containing membrane may be used as a carrier for biologically active substances, as described above, and may be added ex tempore and/or incorporated to the product by absorption. Alternatively, the biologically active substance may be incorporated into the manufacturing process for the collagen-containing membrane by, for example, incorporation into the aqueous dispersion of natural polymer, which dispersion is later formed into the membrane or sponge by air-drying or lypholisation, respectively. The thermal compression parameters of temperature, pressure, moisture content and timing are selected to preserve the biologic activity of the particular additive, and at the same time impart desired characteristics to the resulting compressed material. If used for a medical implant, such preferred properties include drug elution rate, biodegradation rate, and the ability to avoid the development scar tissue buildup at the site of implant insertion.

The thermally compressed material of this invention may be packaged using any suitable packaging and end-sterilized by, for example, ethylene oxide vapors, gamma radiation, electron beam radiation or any other sterilization procedure suitable for such material. Alternatively, before or after sterilization, the compressed membrane according to the present invention may used to manufacture multilayer composites, and/or shaped into various forms of implants or prostheses, for medical and surgical use.

Still another subject of the present invention is the use of the novel material of the invention for the medical indications and applications mentioned above.

The following examples are intended to further illustrate the invention.

### EXAMPLE 1

Manufacturing of a Collagen-based Membrane-like Material.

A freeze-dried collagen sponge, sold under the trademark, Collatamp® (manufacturer: SYNTACOLL AG, Herisau, Switzerland), having a thickness of 5 mm and a collagen content of 5.6 mg/cm³ is conditioned in a moisture chamber to a moisture content of 14% by weight. After conditioning, a mechanical pressure of 5 kg/cm² is applied continuously to both sides of the sponge for 10 seconds. The temperature of pre-heated press surfaces is 80° C. and remains constant during pressing. After such thermal pressing procedure (ThermPress™), the resulting paper-like collagen-membrane has a thickness of 0.1 mm.

Physical properties of the newly created product are markedly improved, if compared to a standard collagen sponge: (1) the product is easy to handle and may be rolled or screwed without breaking, (2) the swelling time of the product is dramatically reduced, (3) the absorption of fluids increases, (3) the wet product (after fluid absorption and/or swelling) remains very flexible, (4) the wet product has much better elasticity and excellent wet tensile strength.

The novel features of the product make it very interesting for use in general surgery, vascular surgery, neurology, and neurosurgery, orthopedics and orthopedic surgery, cardiosurgery, gynecologic surgery, ophthalmology, laryngology, and in all other medical and veterinary disciplines including wound healing and burns. Moreover, the novel product may have benefit if used as a tissue substitute or as a matrix for cell growth especially in tissue engineering and creation of artificial organs.

### EXAMPLE 2

Manufacturing of a Collagen-sheet with Improved Swelling Properties

A freeze-dried collagen-based sponge (collagen content 30 mg/cm³) having a thickness of 5 mm is conditioned in a moisture chamber to a water content of 14% of weight. After conditioning, pressure of 5 kg/cm² and heat are applied simultaneously to both sides of the sponge for 10 seconds. The temperature of pre-heated press surfaces is 80° C. and remains constant during pressing.

After such a thermal pressing procedure (ThermPress™), the resulting collagen sheet is 0.6 mm thick and has the appearance of a strong paper or is leather-like. If compared to standard freeze-dried collagen sponge, such collagen sheet has dramatically improved swelling properties. Moreover, the swelling time is markedly reduced and fluid-binding capacity is increased. Such collagen sheet swells to about 30 times its weight in a maximum time of 10 seconds. As a consequence, the hemostatic properties are markedly increased.

In its swollen condition, the collagen sheet is mechanical stable and has a high wet tensile strength. Both the dry and the wet sheet are very easy to handle, and can be cut to any desired or suitable form. Due to its high stiffness and high elasticity, it is easier to apply it to different locations in the body.

### EXAMPLE 3

Mechanical Strength Determination

This example measures the wet tensile strength, and suture retention limits, of collagen sheets of the present invention.

The collagen layers are prepared from reconstituted collagen sponges (Collatamp®, 10×10 cm) that have not been subjected to sterilization procedures, and that have been stored at room temperature and 13% relative humidity. Collatamp® sponges are prepared from an aqueous dispersion of 0.4% bovine collagen, include about 5.6 mg/cc collagen and have a dry thickness of about 5 mm. These sponges have a spongy, porous surface and smooth skin surface. Single layers are covered on their press plate sides with medical grade paper, placed in a hydrostatic press (Vogt GmbH, Berlin, Germany) pre-calibrated to 80 degrees C., and subjected to a uniform mechanical pressure of 5 kg/cm² (23 bar for 9.5×9.5) for 30 seconds.

Materials tested are (1) uncompressed single layers of reconstituted collagen sponge (Collatamp®) and reconstituted collagen transparent membrane (Collatamp Fascie_), and (2) compressed single layers of reconstituted collagen sponge (Collatamp®).

Test Methodology:

(1) Wet tensile strength: Test strips (8 mm×20 mm) of the samples are allowed to swell for at least 5 minutes in deionized water. A piece of cardboard is insert between the rubberized jaws of the universal testing machine and an initial pulling force of 0.2 newtons (N) applied to stretch the sample at a pulling speed of 60 mm/minute.

(2) Suture retention: Test strips (15 mm×30 mm) marked 5 mm from the edge of the small side and 7.5 mm from the edge of the long side. The test strips are allowed to swell for at least 5 minutes in deionized water. The unmarked small ends of the test strips are inserted (together with the layer of cardboard) into the jaws of the testing machine. A suture (Certilen EP 2, DS 18, braided thread, 0.2-0.25 mm, needle: 18 mm) is drawn through the mark, attached to the lower jaw, and the tear through force for the suture is determined. Each sample is analyzed five times. The initial pulling force is 0.1 newtons (N) and the sample is stretched at a speed of 60 mm/minute

The result of this testing is presented below in Table 1.

TABLE 1

| Sample | Thickness | Wet tensile strength | Suture retention |
|---|---|---|---|
| No. | (mm) | (N) (8 mm) | (max) (N) |
| 0 * | 4.9 | 1.75 | 0.32 |
| 1 | 0.12 | 2.20 | 0.41 |
| Fascie | 0.03 | 2.87 | 0.22 |

* Not compressed

The wet tensile strength of the compressed Collatamp sponge is higher than that of the uncompressed sponge, and is comparable to that of Collatamp Fascie. The suture retention of the compressed sponge is improved relative to the uncompressed sponge and the Collatamp Fascie membrane. After puncturing by the needle, the Collatamp Fascie suffers from widespread and easy irregular tearing. The compressed sponge is much less sensitive in this regard and tears more uniformly.

### EXAMPLE 11

Physical and Fluid Absorption Measurements of Compressed Reconstituted Materials

Single layers of EO-sterilized and non-sterile Collatamp® sponges are compressed and their physical properties measured. Collatamp® sponges (10×10 cm) that have, and have not, been sterilized are removed from their packaging and conditioned for 1 hour at 25° C. and 50% r. h. in an environmental test chamber (Binder). Based on a dry weight determination, the moisture content of the samples is 17.4% for the non-sterile sponges and 14.4% for the ethylene-oxide sterilized sponges. Two sponges of the same type are placed in a medical grade paper bag protected by a plastic bag and the protected construct placed into an environmental chamber. The time between removal from the environmental test chamber and thermal compression is kept as short as possible. The single layer sponges are compressed in a hydrostatic press (Vogt) pre-calibrated to different temperatures (30° C., 40° C., 60° C., 80° C., 100° C., 120° C., 140° C., 160° C.) at uniform mechanical pressures of 5 kg/cm² for 10 seconds, or 10 kg/cm2 for 10 seconds. The EO-sterilized compressed sponges are compressed only at 5 kg/cm2.

The compressed samples are weighed dry, then completely immersed in deionized water at room temperature and allowed to swell for a one-minute or one hour. The samples are retrieved vertically with two pairs of forceps and allowed to drain for 5 sec. The lower edge is stripped of water and weighed.

The thickness, dry and wet, and the amount of water absorbed by the compressed sponges are presented in Tables 2A and 2B below.

TABLE 2A
EO-Sterile Collatamp Sponge Layers (5 kg/cm2)

| | | | | | Water | | | |
|---|---|---|---|---|---|---|---|---|
| Com- | | | | Wet | uptake | | Wet | Water |
| pression | Dry | Wet | Dry | Mass 1 | (mg/ | Dry | Mass 2 | uptake |
| temp | Thick- | Thick- | Mass | (60 sec) | mg) | mass 2 | (60 min) | (mg/mg) |
| (° C.) | ness | ness | 1 | (grams) | (60 sec) | (mg) | (Grams) | (60 min) |
| 30 | 0.16 | 0.36 | 72 | 0.40 | 4.6 | 72 | 0.69 | 8.6 |
| 40 | 0.13 | 0.35 | 87 | 0.50 | 4.7 | 87 | 0.74 | 7.5 |
| 60 | 0.10 | 0.30 | 78 | 0.40 | 4.1 | 82 | 0.57 | 6.0 |
| 80 | 0.09 | 0.21 | 73 | 0.35 | 3.8 | 83 | 0.54 | 5.5 |
| 100 | 0.06 | 0.23 | 79 | 0.41 | 4.2 | 77 | 0.50 | 5.5 |
| 120 | 0.06 | 0.21 | 82 | 0.45 | 4.5 | 85 | 0.59 | 5.9 |
| 140 | 0.06 | 0.23 | 73 | 0.42 | 4.8 | 68 | 0.43 | 5.3 |
| 160 | 0.05 | 0.33 | 86 | 0.45 | 4.2 | 76 | 0.58 | 6.6 |
| 180 | 0.05 | 0.37 | 71 | 0.36 | 4.1 | 72 | 0.58 | 7.1 |
| 200 | 0.05 | 0.22 | 72 | 0.37 | 4.1 | 77 | 0.53 | 5.9 |

TABLE 2B
Non-Sterile Collatamp Sponge Layers (5 kg/cm2) and 10 kg/cm2)

| | | | | | | | | Water |
|---|---|---|---|---|---|---|---|---|
| | | | | | Water | | | uptake |
| Com- | | | | Wet | uptake | | Wet | (mg/ |
| pression | Dry | Wet | Dry | Mass-1 | (mg/ | Dry | Mass-2 | mg) |
| temp | Thick- | Thick- | Mass- | (60 sec) | mg) | mass-2 | (60 min) | (60 |
| (° C.) | ness | ness | 1 | (grams) | (60 sec) | (mg) | (Grams) | min) |

Pressure 5 kg/cm²

| | | | | | | | | |
|---|---|---|---|---|---|---|---|---|
| 30 | 0.17 | 0.39 | 79 | 0.36 | 3.6 | 93 | 0.80 | 7.6 |
| 40 | 0.17 | 0.33 | 81 | 0.38 | 3.7 | 93 | 0.77 | 7.3 |
| 60 | 0.10 | 0.24 | 82 | 0.41 | 4.0 | 86 | 0.68 | 6.9 |
| 80 | 0.08 | 0.22 | 79 | 0.40 | 4.1 | 87 | 0.63 | 6.2 |
| 100 | 0.07 | 0.23 | 78 | 0.47 | 5.0 | 81 | 0.61 | 6.5 |
| 120 | 0.05 | 0.18 | 78 | 0.39 | 4.0 | 85 | 0.60 | 6.1 |
| 140 | 0.05 | 0.18 | 78 | 0.34 | 3.4 | 76 | 0.51 | 5.7 |
| 160 | 0.06 | 0.25 | 72 | 0.39 | 4.4 | 83 | 0.61 | 6.3 |

Pressure 10 kg/cm²

| | | | | | | | | |
|---|---|---|---|---|---|---|---|---|
| 30 | 0.12 | 0.29 | 80 | 0.33 | 3.1 | 89 | 0.62 | 6.0 |
| 40 | 0.11 | 0.22 | 80 | 0.40 | 4.0 | 92 | 0.63 | 5.8 |
| 60 | 0.08 | 0.21 | 85 | 0.39 | 3.6 | 87 | 0.53 | 5.1 |
| 80 | 0.06 | 0.21 | 79 | 0.34 | 3.3 | 85 | 0.55 | 5.5 |
| 100 | 0.06 | 0.22 | 85 | 0.40 | 3.7 | 82 | 0.48 | 4.9 |
| 120 | 0.07 | 0.22 | 77 | 0.39 | 4.1 | 80 | 0.53 | 5.6 |
| 140 | 0.06 | 0.21 | 76 | 0.44 | 4.8 | 80 | 0.54 | 5.8 |
| 160 | 0.05 | 0.20 | 70 | 0.38 | 4.4 | 87 | 0.51 | 4.9 |

The samples compressed with 5 kg/cm² remain opaque white up to a temperature of 160° C., while the samples compressed with 10 kg/cm² assume more of a parchment-like transparent appearance at temperatures of 100° C. and above. With increasing temperature, the compressed sponge samples thickness is increasingly reduced.

### EXAMPLE 4

Physical Measurements of Compressed Denser Collagen Sponges

(1) Single Sponge Measurement Processed under Optimized Conditions

Collatamp® II sponges are prepared from a dispersion of 2.5% bovine collagen, and have a density of about 30 mg/cc collagen. The Collatamp® II sponges used in this experiment include about 15% water content based on dry weight and are not sterile.

The Collatamp II sponges are preconditioned and compressed as described above for the Collatamp® sponges at a pressure of 10 kg/cm² and a temperature of 80° C. for 10 seconds. The compressed sponges are immersed in deionized water and the time for their complete swelling observed. The thermally compressed sponge was completely expanded in about 10 seconds, in comparison with an uncompressed Collatamp II sponge that took about two minutes to completely swell. Measurements of the water swelling behavior of the compressed and uncompressed Collatamp II sponges after ten and 90 seconds are presented in Table 3 below.

TABLE 3

| | Collatamp II | | | |
|---|---|---|---|---|
| | | | Thermal | |
| Test Sample | Uncompressed | | Compressed | |
| Swelling time (sec) | 10 | 90 | 10 | 90 |
| Sample weight (mg) | 335 | 355 | 343 | 326 |
| Water uptake (g) | 5.18 | 8.71 | 10.10 | 9.36 |
| Water uptake (mg/mg of sample) | 15.5 | 24.5 | 29.4 | 28.7 |
| Water uptake (% of max) | 53 | 84 | 100 | 100 |

(2) Properties of Compressed Dense Sponges Processed Under Varied Conditions

This example measures the physical properties of Collatamp® II sponges processed at various temperatures and pressures of 5 kg/cm² and 10 kg/cm².

Preparation of the samples: Collatamp® II sponges (10×10 cm) prepared from a dispersion of 2.5% equine collagen, having a density of about 30 mg/cc collagen and that have, and have not, been sterilized, are removed from their packaging and conditioned for 2 hours at 25° C. and 50% r. h. in an environmental test chamber (Binder). Based on a dry weight determination, the moisture content of the samples is between about 15.2 and 16.6%. Two sponges are placed in a medical grade paper bag protected by a plastic bag and the protected material placed into an environmental chamber. The time between removal from the environmental test chamber and thermal compression is kept as short as possible. The single layer sponges are compressed in a hydrostatic press (Vogt GmbH, Berlin, Germany) pre-calibrated to different temperatures (30° C., 40° C., 60° C., 80° C., 100° C., 120° C., 140° C., 160° C.) at uniform mechanical pressures of 5 kg/cm² for 10 seconds, or 10 kg/cm2 for 10 seconds. The compressed samples are weighed dry, then completely immersed in deionized water at room temperature and allowed to swell for a ten seconds or one hour before measurement. The samples are retrieved vertically with two pairs of forceps and allowed to drain for 5 sec. The lower edge is stripped of water and weighed.

The thickness of the compressed sponge, dry and wet, and the amount of water abosrded presented in Table 4 below.

TABLE 4

| Collatamp II Sponge Layers (5 kg/cm2) and 10 kg/cm2) | | | | | | | | Water |
|---|---|---|---|---|---|---|---|---|
| | | | | | Water | | | uptake |
| Com- | Dry | Wet | Dry | Wet | uptake | | Wet | (mg/ |
| pression | Thick- | Thick- | Mass | Mass 1 | (mg/ | Dry | Mass 2 | mg) |
| temp | ness | ness | 1 | (10 sec) | mg) | mass 2 | (60 min) | (60 |
| (° C.) | (mm) | (mm) | (g) | (grams) | (10 sec) | (g) | (grams) | min) |
| Not | 5.0 | 4.5 | 0.40 | 8.97 | 21.4 | 0.38 | 12.22 | 31.2 |
| com- | | | | | | | | |
| pressed | | | | | | | | |

Pressure = 5 kg/cm²

| | | | | | | | | |
|---|---|---|---|---|---|---|---|---|
| 30 | 2.2 | 4.5 | 0.32 | 7.77 | 23.3 | 0.38 | 12.00 | 30.6 |
| 40 | 1.5 | 4.5 | 0.33 | 8.30 | 24.2 | 0.41 | 12.65 | 29.9 |
| 60 | 0.9 | 4.5 | 0.37 | 10.10 | 26.3 | 0.38 | 12.16 | 31.0 |
| 80 | 0.45 | 3.8 | 0.36 | 9.15 | 24.4 | 0.37 | 11.69 | 30.6 |
| 100 | 0.32 | 3.0 | 0.35 | 3.09 | 7.8 | 0.35 | 9.24 | 25.4 |
| 120 | 0.23 | 0.70 | 0.36 | 1.22 | 2.4 | 0.39 | 1.75 | 3.5 |
| 140 | 0.26 | 0.80 | 0.35 | 1.61 | 3.6 | 0.31 | 2.10 | 5.8 |
| 160 | 0.25 | 0.85 | 0.33 | 1.70 | 4.2 | 0.40 | 2.42 | 5.1 |
| 180 | 0.27 | 1.30 | 0.38 | 2.29 | 5.0 | 0.40 | 3.05 | 6.6 |
| 200 | 0.24 | 0.80 | 0.30 | 1.45 | 3.8 | 0.38 | 1.94 | 4.1 |

Pressure = 10 kg/cm²

| | | | | | | | | |
|---|---|---|---|---|---|---|---|---|
| 30 | 1.80 | 4.5 | 0.35 | 10.35 | 28.6 | 0.38 | 12.57 | 32.1 |
| 40 | 1.10 | 4.5 | 0.33 | 9.00 | 26.3 | 0.36 | 11.17 | 30.0 |
| 60 | 0.60 | 4.5 | 0.33 | 7.68 | 22.3 | 0.32 | 10.09 | 30.5 |
| 80 | 0.30 | 3.5 | 0.36 | 7.41 | 19.6 | 0.33 | 7.98 | 23.2 |
| 100 | 0.28 | 1.0 | 0.33 | 1.29 | 2.9 | 0.35 | 2.27 | 5.5 |
| 120 | 0.26 | 0.65 | 0.33 | 1.09 | 2.3 | 0.40 | 1.91 | 3.8 |
| 140 | 0.20 | 0.55 | 0.35 | 0.74 | 1.1 | 0.35 | 1.45 | 3.1 |
| 160 | 0.18 | 0.50 | 0.32 | 0.95 | 2.0 | 0.30 | 1.34 | 3.5 |
| 180 | 0.17 | 0.50 | 0.30 | 0.72 | 1.4 | 0.33 | 1.28 | 2.9 |
| 200 | 0.17 | 0.55 | 0.34 | 0.71 | 1.1 | 0.37 | 1.41 | 2.8 |

Sponges that are compressed with 5 kg/cm² at a temperature of 140° C. and above begin to assume a parchment-like condensed appearance with vitreous spots. Sponges that are compressed at 10 kg/cm² a temperature of 120° C. and above assume a similar appearance. Beginning with 140/160° C. the swollen samples have a leathery consistency and the sponges' color changes from white to yellow.

Water swelling capacity at 10 seconds decreases rapidly for samples prepared at 10 kg/cm2 beginning with a temperature of 100°0 C. The one-hour water absorption capacity of samples prepared at 100° C. (Skg/cm2) reflect slower water absorption, although the samples absorb at least about 75% of the water absorbed by the uncompressed sponge in one hour. Above 100° C., the one-hour capacities correlate to the 10-second capacity. At and below these temperature and pressure parameters, the compressed sponges are capable of recovering at least two thirds of their original thickness.

### EXAMPLE 5

Biological Properties—Collatamp II

This example measures the biological properties, and specifically the hemostatic relevant properties, of Collatamp® II sponges processed at various temperatures and a pressure of 10 kg/cm². The aggregation of platelets is measured to determine the hemostatic properties and extent, if any, of collagen denaturation present in the processed sponges. A delay in platelet aggregation is believed to correspond to the presence of amounts of denatured collagen.

Preparation of the samples: Collatamp® II sponges (10×10 cm) that have, and have not, been sterilized, are removed from their packaging and conditioned for 2 hours at 25° C. and 50% r.h. in an environmental test chamber (Binder). Based on a dry weight determination, the moisture content of the samples is 15.2%. Two sponges are placed in a medical grade paper bag protected by a plastic bag and the protected material placed into an environmental chamber. The time between removal from the environmental test chamber and thermal compression is kept as short as possible. The single layer sponges are compressed in a hydrostatic press (Vogt) pre-calibrated to different temperatures (30° C. 40° C. 60° C. 80° C. 100° C. 120° C. 140° C., 160° C.) at uniform mechanical pressures of 10 kg/cm2 for 10 seconds.

Measurement of Platelet Aggregation: A sample of the compressed sponge is shredded and homogenized. A measured portion (about 29.4 mg) is dispersed in sterile aqueous buffer and an amount of dispersion providing 10 μg (1 ml) introduced into the Aggregometer (APACT). Samples that exhibit reduced platelet aggregation are retested using a 50-μg (1 ml) sample. The standard solubilized collagen reference is a 1-μg sample results in 89% aggregation in 46 seconds. The results of the testing are presented in Table 5 below.

TABLE 5
Collatamp II-Platelet Aggregation
(Thermally Compressed)

| Compression | Homogenized | Aggregation | | Aggregation | |
|---|---|---|---|---|---|
| temperature | Sample | (10 μg) | | (10 μg) | |
| (° C.) | Weight (mg) | % of Max | T (sec) | % of Max | T (sec) |
| Un- | 29.4 | 87 | 46 | — | — |
| compressed | | | | | |
| 30 | 29.6 | 86 | 82 | — | — |
| 40 | 29.3 | 86 | 61 | — | — |
| 60 | 29.8 | 88 | 68 | — | — |
| 80 | 29.0 | 89 | 67 | — | — |
| 100 | 29.6 | 86 | 64 | 89 | 43 |
| 120 | 29.1 | 86 | 96 | 91 | 55 |
| 140 | 29.6 | n.a. | — | 88 | 91 |

Samples processed at temperatures at and below 120° C. are finely homogenized. Beginning with the sample prepared at 140° C., a fine homogenization and thus even distribution in the liquid is not possible; these samples are increasingly dispersed in the form of large particles.

Up to a compression temperature of 100° C., no negative effect on platelet aggregation is observed. The samples processed at 120° C. exhibit the first signs of a visible delay in the aggregation, while aggregation is still detectable with 50 μg samples prepared at 140° C. Consequently, samples prepared at 140° C. still retain collagen in substantially native form. The altered homogenization properties of these higher temperature-processed samples, which altered properties are observed during sample preparation, correlate to the observations of delayed platelet aggregation. Therefore, the test prcedure, which is designed for testing solubilized collagen, reflects delays in aggregation based on particle size differences among samples.

### EXAMPLE 6

Biological Properties—Collatamp I

Single thermally compressed layers of Collatamp-I membranes, containing doxycycline antibiotic, are tested for platelet aggregation. The standard collagen control material, exhibits the following aggregation results: Aggregation: 89.6% (at 33.6 sec); 90.8% (at 35.6 sec); 85.4% (at 53.4 sec)

A chip of the Collatamp-I membrane (PerioColl_) consists of 1.06 mg equine collagen compressed from a 0.28 w/v % collagen sponge (1.0 cm thick) matrix at 8° C., 10 kg/cm2, for 10 seconds, and including 0.5 mg per chip (5×4 mm) of doxycycline.

(A) Non-sterile Chips: Twelve non-sterile chips are suspended in 5 ml of de-ionized water and homogenized. A collagen suspension consisting of 10 μg collagen per 20 μl buffer is tested for platelet aggregation with following results: Aggregation—first test: 95.3% in 33.8 sec; second test: 94,0% in 30,4 sec.

(B) Sterile Chips: Twelve gamma-sterilized (25 kGy) chips are suspended in 5 ml of de-ionized water and homogenized. A collagen suspension consisting of 10 g collagen per 20 μl buffer is tested for platelet aggregation with following results: Aggregation—first test: 93.3% in 71.8 sec; second test: 91.4% in 82.0 sec.

In both sterile and non-sterile collagen tests, the collagen retained its typical platelet aggregation ability. Although the gamma-sterilized material exhibited a delay in the maximal aggregation time, this is known for gamma sterilized collagen materials in general and is gamma dose-dependent.

Thermally compressed sponges demonstrate excellent haemostatic ability that is in specification limits for good hemostats.

The features of the materials prepared according to this invention are useful in haemostasis applications and are superior to all standard hemostatic agents such as standard collagen sponges, gelatin sponges, regenerating cellulose, cotton gaze, etc. Moreover, the product is beneficial in general surgery, vascular surgery, neurology and neurosurgery, orthopedics and orthopedic surgery, cardiosurgery, gynecologic surgery, ophthalmology, laryngology, and in all other medical and veterinary disciplines including wound healing and burns. Moreover, the product is beneficial in tissue substitute and as cell growth matrix applications such as for tissue engineering and creation of artificial organs.

We claim:

1. A biocompatible membrane comprising a reconstituted matrix of natural polymer fibers and fibrils in their native or re-natured form, wherein said membrane is less than one mm in thickness, has a polymer density of about 250 mg/cm3 to about 500 mg/cm3, and is capable of expanding in contact with fluids to form a matrix capable of promoting cell growth.

2. A membrane according to claim 1 wherein said expanded matrix is porous and capable of promoting formation of granulation tissue, angiogenesis, vascularization and epithelization.

3. A membrane according to claim 2 wherein said natural polymer exhibits the hemostatic and non-antigenic properties of native collagen.

4. A membrane according to claim 3, wherein said membrane is capable of absorbing from about 3 to about 30 times its weight in fluids.

5. A membrane according to claim 4, wherein said membrane is capable of absorbing from about 3 to about 10 times its weight in fluids

6. A membrane according to claim 4, wherein said membrane is capable of absorbing from about 20 to about 30 times its weight in fluids

7. A membrane according to claim 4, wherein said membrane is capable of absorbing said fluids in less than about one hour.

8. A membrane according to claim 4 wherein said membrane comprises a compressed sponge of reconstituted collagen.

9. A membrane according to claim 8 having a wet tensile strength useful in surgical suturing.

10. A membrane according to claim 8 having a thickness of about 0.01 mm to about 0.9 mm.

11. A membrane according to claim 9 having a thickness of about 0.05 mm to about 0.1 mm.

12. A membrane according to claim 1 prepared by applying pressure and heat to a natural polymer sponge having a thickness of about 5 mm and a density of about 5 mg/cm3 to about 50 mg/cm3.

13. A membrane according to claim 1 prepared by reducing the thickness of a natural polymer sponge to about 1 percent to about 30 percent of its original thickness.

14. A membrane according to claim 13 prepared by reducing the thickness of a natural polymer sponge to about 1 percent to about 15 percent of its original thickness.

15. A membrane according to claim 14 prepared by increasing the density of a natural polymer sponge by about 8 to about 50 times of its original density.

16. A collagen membrane according to claim 14 prepared by reducing the thickness of a collagen sponge to about 1 percent to about 3 percent of its original thickness, and is capable of absorbing about 3 to about 7 times its weight in fluids in about one hour.

17. A collagen membrane according to claim 12 prepared by reducing the thickness of a collagen sponge to about 5 to about 30 percent of its original thickness, and is capable of absorbing about 4 to about 30 times its weight in fluids in about one hour.

18. A collagen membrane according to claim 17 prepared by reducing the thickness of a collagen sponge to about 6 to about 25 percent of its original thickness, and is capable of absorbing about 25 to about 30 times its weight in fluids in about one hour.

19. A collagen membrane according to claim 1, comprising a sponge or a membrane prepared from a dispersion or suspension containing 0.5-5 weight/volume % of collagen.

20. A process for the preparation of a biocompatible membrane comprising a reconstituted matrix of natural polymer fibers and fibrils and having improved mechanical and fluid absorption properties, said process comprising applying pressure and heat simultaneously to a natural polymer sponge to reduce the thickness of said sponge to about 1 to about 30 percent of the thickness of said sponge, for a time sufficient to improve said mechanical properties and to preserve the native and/or re-natured form of said fibers and fibrils.

21. A process according to claim 20, wherein said pressure is about 0.1 to about 1000 kg/cm2, said elevated temperature is about 50 to about 200 degrees C., and said time is about 0.1 to about 60 seconds.

22. A process according to claim 21, wherein said pressure is about 5 to about 25 kg/cm2, said elevated temperature is about 40 to about 140 degrees C., and said time is about 0.1 to about 60 seconds.

23. A process according to claim 22 wherein said pressure is applied for about 5 to about 10 seconds.

24. A process according to claim 22 wherein said temperature is about 80 to about 140 degrees C.

25. A process according to claim 20 wherein the density of said sponge is between about 2 to about 60 mg/cc and has a thickness of about 1 to about 10 mm.

26. A process according to claim 25 wherein the density of said sponge is between about 6 to about 30 mg/cm3.

27. A process according to claim 25 wherein said membrane has a density of about 250 mg/cm3 to 300 mg/cm3.

28. A process according to claim 20 wherein said improved mechanical properties comprise dry and wet tensile strength, suturing and wetting ability, and flexibility such that said membrane is capable of being rolled, screwed in both dry and wet condition, cut and meshed without breaking or deforming.
